(19) United States
(12) Patent Application Publication
(10) Pub. No.: US 2012/0180793 A1
(43) Pub. Date: Jul. 19, 2012

(54) Dynamic Graphic Respiratory Communication System

(76) Inventor: Ben J. Schoepke, Madison, WI (US)

(21) Appl. No.: 13/329,219

(22) Filed: Dec. 17, 2011

Related U.S. Application Data
(60) Provisional application No. 61/424,306, filed on Dec. 17, 2010.

(51) Int. Cl. A61M 16/00

(52) U.S. Cl. 128/204.22

(57) ABSTRACT

The present application is directed toward a dynamic graphic communication system for use with various graphical user interfaces provided on the displays of anesthesia machines and other medical systems. Instead of using traditionally static icon, dynamic graphics are used to provide additional, intuitive information, including real-time information regarding the functioning of various components of an anesthesia machine, such as ventilator system, vaporizers and patient parameter monitors.

### CROSS-REFERENCE TO RELATED APPLICATIONS

The present specification claims priority from U.S. Provisional Patent Application No. 61/424,306, entitled “Animated Display Icons for Use in Anesthesia Systems” and filed on Dec. 17, 2010, which is herein incorporated by reference in its entirety.

### FIELD

This invention relates generally to anesthesia systems, ventilators and patient monitors for use in operating rooms, intensive care and home care settings. More particularly, the present invention relates to dynamic functional and informational graphics projected on anesthesia machine displays.

### BACKGROUND

Anesthesia systems are used in operating rooms and intensive care units to supply a continuous and accurate mixture of medical gases and anesthetic agents to patients in order to induce loss of physical sensation, particularly pain, and to keep the patients sedated during procedures and in critical situations. Typical anesthesia systems include an anesthesia machine connected to hospital piped gases. Anesthesia machines are mounted on wheels and usually comprise a ventilator, one or more vaporizers for addition of volatile anesthetic agents, a suction unit, patient monitoring devices, displays, and a work bench. The machines are typically positioned in proximity to the head of the patient, allowing enough room for surgeons and nurses to operate and maneuver. Anesthesiologists and/or nurse anesthetists stand by the machine at the head of the patient and are able to observe both the patient and the anesthesia machine displays, enabling them to react quickly if an emergency situation arises.

Modern anesthesia machines include one or two displays that can be viewed by the anesthetist. The first display relays information regarding the functionality of the ventilator. State of the art software development packages have made the use of small icons an expected and easily understood component of user interfaces on touchscreen anesthesia machine displays. For example, the icon of a bellows is known to represent ventilation delivery. Traditionally, such icons are static and when pressed allow the user to access submenus. In the above example, pressing the bellows icon would provide the user quick access to ventilation setting menus. A second display is often included and typically relays physiological data regarding the status of the patient obtained from the patient monitoring devices of the anesthesia machine. This data is displayed via numerical and waveform representation and often includes parameters such as heart rate, respiratory rate, blood pressure, and oxygen saturation.

Though the icons described above for the first display provide the user with quick access to system settings, they do not relay real-time information regarding the operational status or functionality of the anesthesia system. For example, a static icon of a bellows does not tell the user whether or not the ventilation is switched on or at what level the ventilation is delivering gases. Therefore, a need exists for a communication system which uses graphics dynamically to provide quick access to system settings and also to quickly relay system information to the user (anesthetist/clinician). In addition, such a communication system would enhance the user's intuitive understanding of each individual icon's purpose.

### SUMMARY

The present specification is directed toward a ventilator system, which, in one embodiment, has at least one processor, a gas supply, a breathing circuit, a display, and a volatile or non-volatile computer readable medium, not including transmission media for transmitting waves for storing a plurality of programmatic instructions, wherein, when said programmatic instructions are executed by said at least one processor, the system: monitors a volume of gas being delivered by said breathing circuit to a patient; displays a graphical image comprising a cylinder having expandable folds; based on said monitoring, determines an amplitude of said cylinder having expandable folds; displays a modified cylinder having expandable folds wherein said folds are in a state of compression or expansion depending upon said determined amplitude; determines, on a real-time basis, a rate for said delivery of the gas to the patient; and displays, on a real-time basis, a modified cylinder having expandable folds wherein said folds are in a state of compression or expansion depending upon said rate of gas delivery.

Further, the cylinder has a maximum amplitude and wherein, in a state of said maximum amplitude, a peak inspiration by the patient is detected and the folds are fully expanded. Still further, the cylinder has a minimum amplitude and wherein, in a state of said minimum amplitude, a peak expiration by the patient is detected and the folds are fully compressed.

In one embodiment, the dynamic graphic display of the modified cylinder, cycles, in real-time between a fully expanded state and a fully compressed state and wherein said cycle defines a rate of change between said fully expanded state and said fully compressed state. In one embodiment, this rate of change is indicative of the actual rate of inspiration and expiration of the patient.

The present specification also describes a ventilator system having at least one processor, a gas supply, a breathing circuit, and a display, a volatile or non-volatile computer readable medium, not including transmission media for transmitting waves, wherein said computer readable medium comprises: programmatic instructions for monitoring a volume of gas being delivered by said breathing circuit to a patient; programmatic instructions for displaying a graphical image comprising a structure having expandable and compressible folds; programmatic instructions for determining an amplitude of said structure based on said monitoring; programmatic instructions for displaying a modified structure having expandable and compressible folds wherein said folds are in a state of compression or expansion depending upon said determined amplitude; programmatic instructions for determining, on a real-time basis, a rate for delivery of gas to the patient; and programmatic instructions for displaying, on a real-time basis, a modified structure having compressible and expandable folds wherein, said folds are in a state of compression or expansion depending upon said rate of gas delivery.

Further, the cylinder has a maximum amplitude and wherein, in a state of said maximum amplitude, a peak inspiration by the patient is detected and the folds are fully expanded. Still further, the cylinder has a minimum amplitude and wherein, in a state of said minimum amplitude, a peak expiration by the patient is detected and the folds are fully compressed.

In one embodiment, the dynamic graphic display of the modified cylinder, cycles, in real-time between a fully expanded state and a fully compressed state and wherein said cycle defines a rate of change between said fully expanded state and said fully compressed state. In one embodiment, this rate of change is indicative of the actual rate of inspiration and expiration of the patient.

In one embodiment, the fully expanded state of the structure is displayed when the patient is at peak inspiration. In another embodiment, the fully compressed state of the structure is displayed when the patient is at peak expiration.

The aforementioned and other embodiments of the present shall be described in greater depth in the drawings and detailed description provided below.

### BRIEF DESCRIPTION OF THE DRAWINGS

These and other objects and advantages of the present invention will become more fully apparent from the following detailed description when read in conjunction with the accompanying drawings with like reference numerals indicating corresponding parts through-out, wherein:

FIG. 1 is an illustration of an exemplary anesthesia system layout;

FIG. 2A is an illustration of one embodiment of an exemplary dynamic respiratory graphic on the screen of an anesthesia machine display, with the graphic fully inflated, indicative an active state;

FIG. 2B is an illustration of the exemplary dynamic respiratory graphic, as shown in FIG. 2A, with the graphic fully deflated, indicating an inactive state;

FIG. 3A illustrates an embodiment of a work flow for animation of the dynamic respiratory graphic;

FIG. 3B illustrates another embodiment of a work flow for animation of the dynamic respiratory graphic; and

FIG. 3C illustrates yet another embodiment of a work flow for animation of the dynamic respiratory graphic.

### DETAILED DESCRIPTION

In one embodiment, the present specification is directed toward a communication system for use with various graphical user interfaces provided on anesthesia machine displays. Using software and displays, traditionally static icons are animated, resulting in dynamic graphics that portray a higher level of software functionality to the user (anesthetist/clinician), as well as provide additional, intuitive information regarding the graphic's purpose. This, as a result, enhances the usability of the GUI as compared to the static icons used in the prior art.

In one embodiment, animated graphics move or change shape dynamically to relay real-time information regarding the functionality of various components of an anesthesia machine, such as, but not limited to, the ventilator system, vaporizers, patient parameter monitors, etc. The dynamic graphics are further used to display information and status of respiratory and other vital parameters of the patient.

The present invention aims to present information on the display in a manner that is clearer and easily identifiable. The use of dynamic graphics is not just limited to displaying information, but also serves the dual purpose of acting as control icons. That is, system functions may be controlled by, for example, clicking on the dynamic graphic using a mouse connected to the computing device, having an associated pointer displayed on the display screen or by tapping on the dynamic graphic in the case where the display is a touchscreen display.

The present application discloses multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In one embodiment, the present specification is directed toward a ventilator system having at least one processor, a gas supply, a breathing circuit, a display, and a volatile or non-volatile computer readable medium, not including transmission media for transmitting waves for storing a plurality of programmatic instructions, wherein, when the programmatic instructions are executed by the at least one processor, the system monitors a volume of gas being delivered by said breathing circuit to a patient; displays a graphical image comprising a cylinder having expandable folds; and based on said monitoring, determines an amplitude of said cylinder having expandable folds. Further, the system then displays a modified cylinder having expandable folds wherein the folds are in a state of compression or expansion depending upon said determined amplitude and determines, on a real-time basis, a rate for said delivery of the gas to the patient; and displays, on a real-time basis, a modified cylinder having expandable folds wherein the folds are in a state of compression or expansion depending upon the rate of the gas delivery.

In another embodiment, the present specification is directed toward a ventilator system having at least one processor, a gas supply, a breathing circuit, and a display, a volatile or non-volatile computer readable medium, not including transmission media for transmitting waves, wherein said computer readable medium comprises: programmatic instructions for monitoring a volume of gas being delivered by said breathing circuit to a patient; programmatic instructions for displaying a graphical image comprising a structure having expandable and compressible folds; programmatic instructions for determining an amplitude of said structure based on said monitoring; programmatic instructions for displaying a modified structure having expandable and compressible folds wherein said folds are in a state of compression or expansion depending upon said determined amplitude; programmatic instructions for determining, on a real-time basis, a rate for delivery of gas to the patient; and programmatic instructions for displaying, on a real-time basis, a modified structure having compressible and expandable folds wherein, said folds are in a state of compression or expansion depending upon said rate of gas delivery.

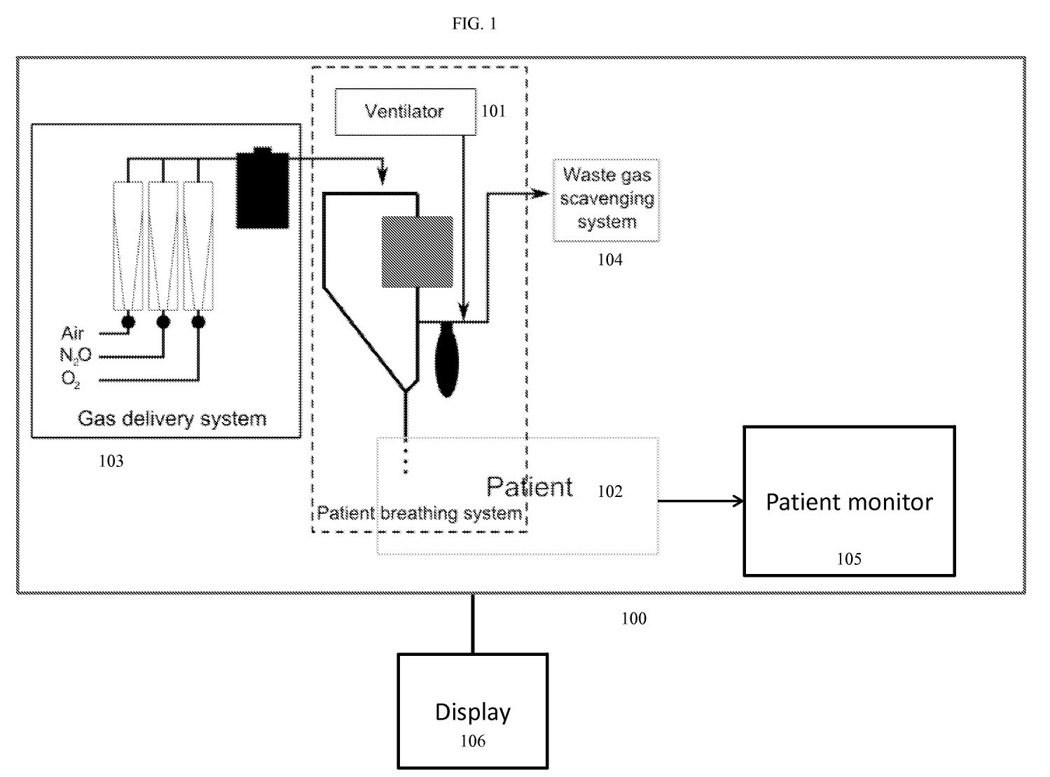

Referring to FIG. 1, an exemplary anesthesia system layout is shown. The system 100 comprises a ventilator 101 for providing assisted respiration to the patient 102. The system 100 further comprises one or more vaporizers 103 for addition of volatile anesthetic agents and gases such as O₂, N₂O and air; a suction unit (not shown); and a scavenging system 104 to remove expired anesthetic gases from the operating room. The anesthesia machine also includes patient monitoring devices 105 for measuring vital parameters of the patient, including, but not limited to heart rate, respiratory rate, blood pressure, oxygen saturation and other parameters that are typically measured while the patient is on a ventilator or under anesthesia.

The system 100 is coupled to at least one display 106, which displays information about the patient parameters and the functioning of the system, by means of a GUI. The GUI also presents various menus that allow users to configure settings according to their requirements. The system 100 further comprises at least one processor (not shown) to control the operation of the entire system and its components. It should further be appreciated that the at least one processor is capable of processing programmatic instructions, has a memory capable of storing programmatic instructions, and employs software comprised of a plurality of programmatic instructions for performing the processes described herein. In one embodiment, the at least one processor is a computing device capable of receiving, executing, and transmitting a plurality of programmatic instructions stored on a volatile or non-volatile computer readable medium.

In one embodiment, the dynamic graphic respiratory communication system (DGRCS) of the present application dynamically and graphically illustrates respiration information. For example, an animated respiratory graphic is presented on the display, and is used to notify the user that the ventilation system is turned on. In one embodiment, the respiratory graphic may move dynamically in time with the ventilation system that is delivering breaths to the patient. In another embodiment, the respiratory graphic assumes an accordion-like cylindrical shape having expandable and compressible folds therein to illustrate that the operational status of the ventilation system. In one embodiment, the folds are in a state of compression or expansion depending upon the determined amplitude of the cylinder. In one embodiment, the dynamic amplitude of the cylinder, based on movement of the respiratory graphic, conveys to the user a measure of the volume of breath being delivered to a patient.

Dynamic graphics indicate to the user the purpose or functionality of each graphic more clearly. For instance, in the exemplary respiratory graphic embodiment described above, the dynamic movement of the graphic intuitively informs the user that this graphic is linked to respiration and may be used to control the ventilator. Dynamic graphics thus relay both system functionality and control options in a manner that is much more intuitive than that in the prior art because the depth of information provided can be used to manipulate system functionality.

The use of dynamic graphics is not just limited to displaying information, but also serves the dual purpose of acting as control icons. That is, system functions may be controlled by, for example, clicking on the dynamic graphic using a mouse connected to the computing device, having an associated pointer displayed on the display screen or by tapping on the dynamic graphic in the case where the display is a touchscreen display.

In various embodiments, the dynamic graphic communication system comprises animation that is configured to accurately represent real-time information. Therefore, some animation is inactivated once the function they represent has been deactivated or switched off. For example, in the embodiment described above, in the exemplary graphic representing respiration, the graphic is deactivated when the ventilator function is turned off. This is used to alert the caregiver as to whether a patient is still under ventilation, thus avoiding user confusion and enhancing usability. Thus, the animation of the dynamic graphic is presented in real-time reflecting the current usage situation.

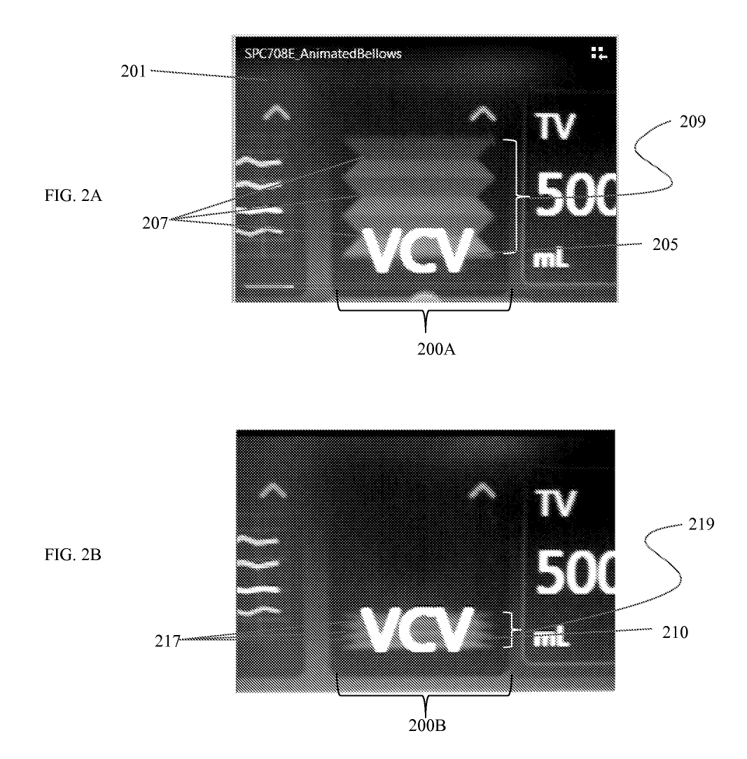

FIG. 2A is an illustration of one embodiment of an exemplary dynamic graphic 200A on the screen 201 of an anesthesia system display. In this embodiment, the dynamic graphic 200A is used to represent the operational status and functionality of the ventilator of the anesthesia system. One of ordinary skill in the art would appreciate that the dynamic graphic may include any symbol, object, picture, figure or icon that represents its purpose suitably. In one embodiment, a dynamic graphic to depict assisted respiration using a ventilator may include the figure of any one of a pump, oxygen cylinder, lungs, bellows, etc. In one embodiment, the shape or figure of a dynamic graphic may be chosen by user from a selection of options presented by the GUI. Regardless of shape or form, the dynamic respiratory graphics move or change shape to illustrate ventilation function. In one embodiment, the size of dynamic respiratory graphics changes to represent ventilation. Referring to FIG. 2A, in one embodiment, the dynamic respiratory graphic 200A is in the shape of an inflatable and deflatable structure, such as cylinder 205 having expansion grooves or expandable folds 207 therein, along the vertical height 209 of the cylinder 205. Thus, as shown in FIG. 2A, the dynamic respiratory graphic 200A includes fully expanded cylinder 205 wherein the expansion grooves or folds 207 are fully extended, i.e. at a maximum height or amplitude. That is, the dynamic respiratory graphic 205 it is at its maximum size. In one embodiment, the maximum amplitude displayed represents when a peak inspiration by the patient is detected.

FIG. 2B is an illustration of the dynamic respiratory graphic shown in FIG. 2A, however, FIG. 2B depicts the dynamic respiratory graphic 200B fully deflated, i.e. at a minimum height or amplitude, such that the cylinder 210 is at its minimum size along the vertical height 219 of the cylinder 210 and expansion grooves or folds 217 are fully compressed. In one embodiment, the minimum amplitude displayed represents when a peak expiration by the patient is detected.

In one embodiment, the height or amplitude (expansion and compression) of the cylindrical graphic to be displayed is determined by measuring the flow going into and out of the bellows components on a ventilator system, and integrating the flows to determine the volume of gas inside the bellows. In another embodiment, the height of the graphic is determined by placing a proximity sensor on the top of the physical bellows canister to determine how far away from the top the physical bellows is away from its enclosure. The proximity sensor would then relay this information to the processing system to display it as an animated graphic as described above.

Thus, in one embodiment, the dynamic respiratory graphic 200 is animated so that it increases and decreases in size (expands and deflates) with each breath provided by the ventilation system of the anesthesia machine. Thus, FIG. 2A depicts a fully expanded cylinder 205 representing dynamic respiratory graphic 200A, showing that a breath is about to be delivered to the patient, while FIG. 2B depicts a modified, fully deflated cylinder 210, representing dynamic respiratory graphic 200B, showing that a full breath has now been delivered to a patient. In one embodiment, the course of each breath supplied by the ventilation system is represented by the cylinder, that is, the dynamic respiratory graphic rising and falling between the fully expanded level and the fully deflated level depicted in FIGS. 2A and 2B, respectively. Thus, on a real-time basis, the cylinder cycles between a fully expanded state and a fully compressed state where the cycle defines a rate of change between the fully expanded state and the fully compressed state. In one embodiment, the fully expanded state of the cylinder is displayed when the patient is at peak inspiration. In one embodiment, the fully compressed state of the cylinder is displayed when the patient is at peak expiration.

In one embodiment, rather than having the dynamic respiratory graphic move to a fully deflated position as shown in FIG. 2B at the conclusion of a breath inspiration delivery, the graphic moves to an intermediate, partly deflated position representative of the amount of volume delivered during a breath. With smaller delivered volumes, the dynamic respiratory graphic would only deflate a small percentage of the full range, while for large delivered volumes it would approach the fully deflated position at the end of the delivered inspiration. Thus, the dynamic respiratory graphic acts as an “at a glance” reference to inform the user that the ventilation system is on and at what level of volume delivery it is functioning.

In one embodiment, the shape of graphic and how it dynamically changes or moves with change in parameters may be customized by users.

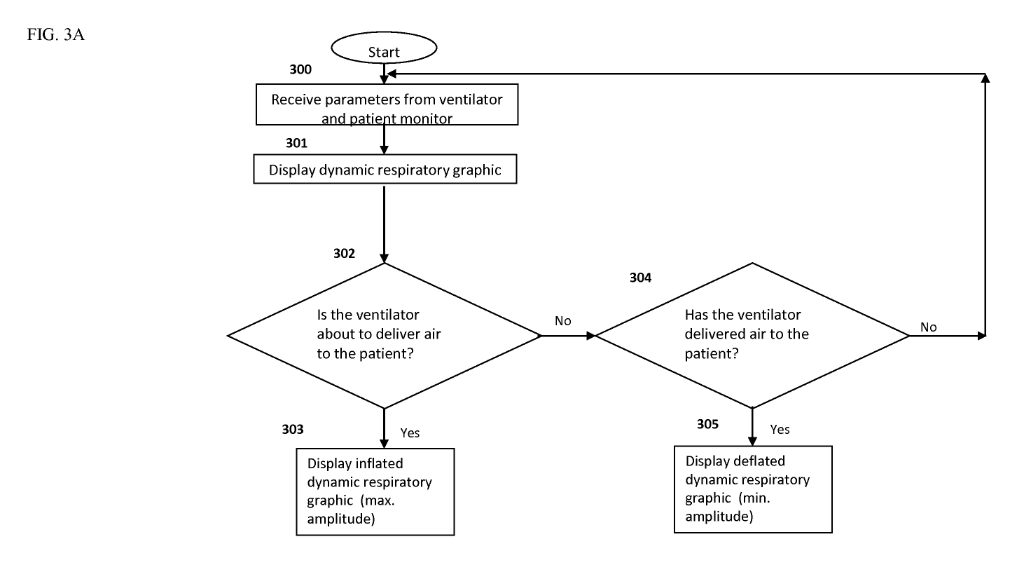

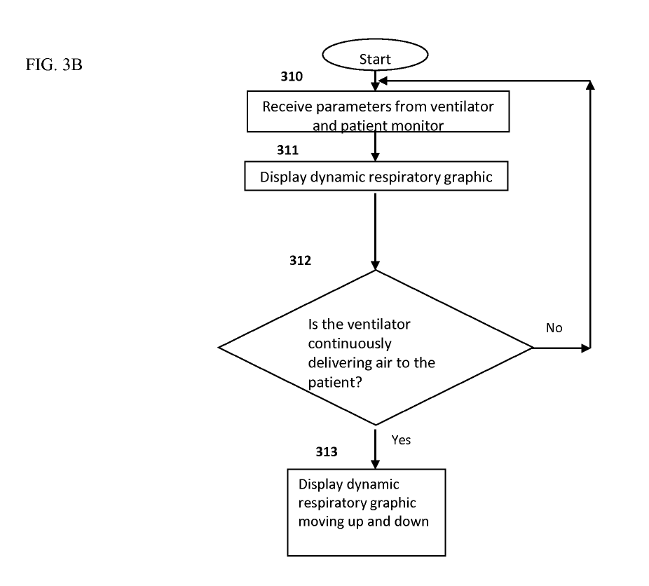

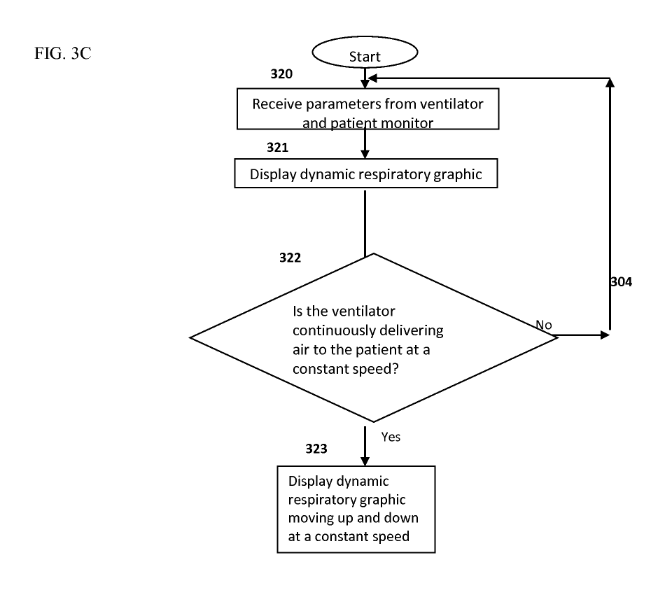

FIGS. 3A, 3B, and 3C illustrate, by means of flowcharts, exemplary process flows for dynamic animation of the respiratory graphic, in response to changing parameters in real time. The following work flows refer to a processor logic which executes appropriate programmatic instructions on receiving parameter data from system components and patient monitors. It should be understood by those of ordinary skill in the art that the system comprises at least one processor to control the operation of the entire system and its components. It should further be appreciated that the at least one processor is capable of processing programmatic instructions, has a memory capable of storing programmatic instructions, and employs software comprised of a plurality of programmatic instructions for performing the processes described herein. In one embodiment, the at least one processor is a computing device capable of receiving, processing, and transmitting a plurality of programmatic instructions stored on a volatile or non-volatile computer readable medium.

Referring to FIG. 3A, on receiving parameter data from the patient monitor and the ventilator, in step 300, the dynamic respiratory graphic is displayed, in step 301, on the screen of the display that is in communication with the system. The processor logic then examines, in step 302, the received parameters to determine if the ventilator is about to deliver air to the patient. The parameters threshold values are, in one embodiment, pre-defined. In another embodiment, the parameter threshold values are pre-set prior to connection to a patient and are dependent upon the patient condition and/or demographics (i.e. neonate, pediatric, adult).

If the ventilator is about to deliver gas/air to the patient, and if the patient is at peak inspiration, then the system displays an expanded dynamic respiratory graphic, that is, the graphic is displayed, in step 303, at its maximum size having a maximum amplitude because the volume of gas being delivered to the patient is at a maximum.

If the ventilator is not about to deliver gas/air to the patient, the system determines if it has already delivered a breath of air, as shown in step 304. In that case, the system displays a modified, compressed dynamic respiratory graphic, that is, the graphic is displayed at its minimum size, having a minimum amplitude and signifying that the patient is at peak expiration, as shown in step 305, because the volume of gas being delivered to the patient is at a minimum.

Thus, the graphic display is dependent upon the rate of gas delivery that is measured by the system and the physical movement of the bellows within the ventilation system.

FIG. 3B illustrates the work flow for another exemplary animation of dynamic respiratory graphic. Referring to FIG. 3B, after receiving patient parameters from the patient monitor and the ventilator, as shown in step 310, the dynamic respiratory graphic is displayed, in step 311, on the system screen. The processor logic then examines the parameters and compares them to threshold values to determine if the ventilator is continuously delivering air to the patient, as shown in step 312. In this case, the dynamic respiratory graphic is displayed as continuously expanding and compressing, to indicate patient inspiration and expiration, respectively, as shown in step 313. The dynamic expansion and compression or vertical movement of the graphic serves to instantly inform the user that the ventilator is operating continuously.

FIG. 3C illustrates the work flow for another exemplary animation of dynamic respiratory graphic. Referring to FIG. 3C, after receiving patient parameters, such as gas volume, and rate of volumetric delivery, from the patient monitor and the ventilator, as shown in step 320, the dynamic respiratory graphic is displayed on the system screen, as shown in step 321. The processor logic then examines the parameters to determine if the ventilator is delivering air to the patient at a constant rate, as shown in step 322. In this case, the dynamic respiratory graphic is displayed as continuously expanding and compressing by moving up and down at a constant rate of speed, which is timed to indicate the rate of the patient's inspiration and expiration, as shown in step 323. From the speed of movement of the dynamic respiration graphic, a user can instantly ascertain the rate at which the ventilator is delivering air to the patient.

In all the above embodiments, the movement or change in the dynamic respiration graphic is in real time response to change in system and patient parameters.

As mentioned earlier, dynamic graphics may be used not only to display status of patient parameters or system parameters, but also to access system menus. Thus, an appropriate dynamic graphic intuitively informs the user which system component it represents and which menus would be available through the particular graphic. For example, in the above described embodiment, the dynamic respiratory graphic instinctively informs the user that the graphic can be used to access the ventilation system's menus and settings.

In another embodiment, the dynamic graphic system is used to relay information obtained by patient monitoring devices to the user in an “at a glance” fashion. For example, in one embodiment, a dynamic graphic is used to display real-time CO₂ waveform information. In one embodiment, the waveform is a small graphic showing a trend or line graph of the CO₂ measurements over time, such as, for example, the last 15 minutes of measurements. In one embodiment, pressing or tapping the graphic as described above, for example, causes the waveform to expand to a larger portion of the display screen. In addition, when expanded, the graphic is capable of presenting more data and/or the real-time CO₂ measurement (i.e. the last 30 seconds of measurements).

In addition, animating the graphic such that it moves or changes shape dynamically to reflect a real-time CO₂ waveform allows the user to grasp CO₂ status instantaneously, without having to permanently sacrifice a larger portion of the display. For example, but not limited to such example, the graphical depiction could be of the patient's lungs wherein the lungs expand and contract to coincide with the actions of the ventilator, with an additional graphical means, such as color change, to indicate the CO₂ levels in the expired gas. In one example, the lung graphic could turn red in color to indicate an increase in CO₂ levels. The display can thus be used to present a larger number of graphics (compared to existing systems), and to reflect a larger amount of information, yet in a lucid manner.

In various embodiments, in addition to anesthesia systems, dynamic graphics can be utilized on displays of independent patient monitoring systems, ICU ventilators, and any other systems instead of static icons to access display or control of real-time information to provide rapid selection of setting or screen control menus, relay information, and for the purpose of providing enhanced intuitive understanding of the graphic and information that it provides.

Thus, in one embodiment, the present invention is a graphical user interface for displaying dynamic graphics pertaining to the functionality of various components of medical systems, and any other systems where icons are used to display and access real-time information. A dynamic graphic provides enhanced understanding of functionality and status of the component it represents. Further, in one embodiment, a user can control and modify the animation of the graphic according to his or her preference.

Although the present invention has been described with reference to an anesthesia system, one of ordinary skill in the art would appreciate that application of dynamic graphics of the present invention may be extended to all kinds of medical monitoring and diagnostic systems that require an interface to present information and seek inputs from users.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A ventilator system having at least one processor, a gas supply, a breathing circuit, a display, and a volatile or non-volatile computer readable medium, not including transmission media for transmitting waves for storing a plurality of programmatic instructions, wherein, when said programmatic instructions are executed by said at least one processor, the system:
monitors a volume of gas being delivered by said breathing circuit to a patient;
displays a graphical image comprising a cylinder having expandable folds;
based on said monitoring, determines an amplitude of said cylinder having expandable folds;
displays a modified cylinder having expandable folds wherein said folds are in a state of compression or expansion depending upon said determined amplitude;
determines, on a real-time basis, a rate for said delivery of the gas to the patient; and
displays, on a real-time basis, a modified cylinder having expandable folds wherein said folds are in a state of compression or expansion depending upon said rate of gas delivery.

2. The ventilator system of claim 1 wherein said cylinder has a maximum amplitude and wherein, in a state of said maximum amplitude, said folds are fully expanded.

3. The ventilator system of claim 1 wherein said processor determines that said cylinder has a maximum amplitude when a peak inspiration by the patient is detected.

4. The ventilator system of claim 1 wherein said cylinder has a minimum amplitude and wherein, in a state of said minimum amplitude, said folds are fully compressed.

5. The ventilator system of claim 1 wherein said processor determines that said cylinder has a minimum amplitude when a peak expiration by the patient is detected.

6. The ventilator system of claim 1 wherein said display, on a real-time basis, of the modified cylinder cycles between a fully expanded state and a fully compressed state and wherein said cycle defines a rate of change between said fully expanded state and said fully compressed state.

7. The ventilator system of claim 6 wherein said rate of change is indicative of the actual rate of inspiration and expiration of the patient.

8. The ventilator system of claim 7 wherein said fully expanded state of the cylinder is displayed when the patient is at peak inspiration.

9. The ventilator system of claim 8 wherein said fully compressed state of the cylinder is displayed when the patient is at peak expiration.

10. In a ventilator system having at least one processor, a gas supply, a breathing circuit, and a display, a volatile or non-volatile computer readable medium, not including transmission media for transmitting waves, wherein said computer readable medium comprises:
programmatic instructions for monitoring a volume of gas being delivered by said breathing circuit to a patient;
programmatic instructions for displaying a graphical image comprising a structure having expandable and compressible folds;
programmatic instructions for determining an amplitude of said structure based on said monitoring;
programmatic instructions for displaying a modified structure having expandable and compressible folds wherein said folds are in a state of compression or expansion depending upon said determined amplitude;
programmatic instructions for determining, on a real-time basis, a rate for delivery of gas to the patient; and
programmatic instructions for displaying, on a real-time basis, a modified structure having compressible and expandable folds wherein, said folds are in a state of compression or expansion depending upon said rate of gas delivery.

11. The ventilator system of claim 10 wherein said structure has a maximum amplitude and wherein, in a state of said maximum amplitude, said folds are fully expanded.

12. The ventilator system of claim 11 wherein said structure has the maximum amplitude only when a peak inspiration by the patient is detected.

13. The ventilator system of claim 10 wherein said structure has a minimum amplitude and wherein, in a state of said minimum amplitude, said folds are fully compressed.

14. The ventilator system of claim 13 wherein said structure has the minimum amplitude only when a peak expiration by the patient is detected.

15. The ventilator system of claim 10 wherein said display, on a real-time basis, of the modified structure cycles between a fully expanded state and a fully compressed state and wherein said cycle defines a rate of change between said fully expanded state and said fully compressed state.

16. The ventilator system of claim 15 wherein said rate of change is indicative of the actual rate of inspiration and expiration of the patient.

17. The ventilator system of claim 16 wherein said fully expanded state of the structure is displayed when the patient is at peak inspiration.

18. The ventilator system of claim 17 wherein said fully compressed state of the structure is displayed when the patient is at peak expiration.
